(19) United States
(12) Patent Application Publication
(10) Pub. No.: US 2025/0248372 A1
(43) Pub. Date: Aug. 7, 2025

(54) UV LED DEVICE FOR TREATING FLUIDS

(71) Applicant: TSLC CORPORATION, CHU-NAN (TW)

(72) Inventors: PO-WEI LEE, HSINCHU COUNTY (TW); YEN-CHAO LIN, TAOYUAN CITY (TW); SHIH-HAI LIU, HSINCHU CITY (TW); SHENG-HO LIU, MIAOLI COUNTY (TW); YU-JU CHEN, CHANGHUA CITY (TW)

(73) Assignee: TSLC CORPORATION, CHU-NAN (TW)

(21) Appl. No.: 19/037,112

(22) Filed: Jan. 25, 2025

Related U.S. Application Data
(60) Provisional application No. 63/627,875, filed on Feb. 1, 2024.

(51) Int. Cl. A01K 63/04; A61L 2/10; A61L 9/20

(52) U.S. Cl. CPC A01K 63/04; A61L 2/10; A61L 9/20; A61L 2202/11

(57) ABSTRACT

A UV LED device for treating a fluid includes a housing configured for submersion in the fluid having a reaction chamber with one or more inlet openings configured to receive untreated fluid and an outlet opening configured to discharge treated fluid. The UV LED device also includes a LED module mounted to the housing configured to emit UV radiation including UV radiation in the UVC wavelength range. The UV LED device also includes a light blocker attached to the housing and a heatsink attached to the housing in thermal communication with the LED module. A UV LED reactor includes a vessel configured to contain a fluid, the UV LED device submerged in the fluid, and a flow generator for maintaining fluid flow paths through the UV LED device. A method for treating a fluid includes providing the UV LED reactor and treating the fluid using the UV LED reactor.

### CROSS REFERENCE TO RELATED APPLICATION

This application claims priority from U.S. Provisional Application No. 63/627,875, filed Feb. 1, 2024, which is incorporated herein by reference.

### FIELD

This disclosure relates to UV (ultraviolet) LED (light emitting diode) devices and particularly to UV LED devices configured to emit UVB and UVC radiation for treating fluids, such as water and air.

### BACKGROUND

A UV LED (light emitting diode) device can be a highly effective tool for treating fluids, such as water and air. For example, UV LED devices are used to treat water for algae control and sterilization, and to treat air for disinfection. The UV radiation emitted by a UV LED device typically has a wavelength range of from 10 nm to 400 nm. Two particularly useful wavelength range of the UV radiation emitted by the UV LED device are UVB radiation having a wavelength range of from 280 nm to 310 nm and UVC radiation having a wavelength range of from 200 nm to 280 nm. For example, UVB and UVC radiation are used in different applications to destroy microorganisms, such as bacteria, viruses, and algae, by disrupting DNA, thus causing harm to healthy cells and preventing reproduction of the microorganisms.

Some examples of applications for UV LED devices configured to emit UVB or UVC radiation include:
1. Algae control for fish tanks and pools: In this application, UVB or UVC radiation effectively inhibits the growth of algae in water. As the water circulates through the fish tank or pool, the UV LED device can be strategically located to expose the water to the UVB or UVC radiation. The UVB or UVC radiation functions to break down the DNA of algae cells, preventing them from multiplying and causing green water issues.
2. Water sterilization: In this application, UV LED devices configured to emit UVB or UVC radiation are effective in sterilizing water by deactivating or destroying bacteria, viruses, and other pathogens. This is particularly important application for drinking water to prevent the spread of diseases.
3. Air disinfection: In this application, UVC LED devices configured to emit UVB or UVC radiation can be integrated into air purification systems to disinfect the air in environments such as homes, offices, and healthcare facilities. This application helps to reduce airborne pathogens, creating a cleaner and healthier indoor air quality.

Referring to FIG. 1, a first prior art UVC reactor 10 for treating water with UVC radiation 14 is illustrated. The UVC reactor 10 includes a UV tube lamp 12 having a UV source 16, such as a low-pressure mercury vapor lamp, configured to emit the UVC radiation 14. The UV tube lamp 12 also includes a quartz sleeve 18, configured to allow the UVC radiation 14 to pass through, while protecting the UV source 16. A reactor chamber 20 contains the UV tube lamp 12 and provides a controlled environment for the water to be exposed to the UVC radiation 14. The reactor chamber 20 includes a water inlet 22 having a water hose 32 in flow communication with a source of untreated water (not shown) and a water outlet 24 in flow communication with a water hose 34 in flow communication with a storage vessel (not shown) for treated water. In the UVC reactor 10, the water flow path is from the water inlet 22 through the reactor chamber 20 to the water outlet 24. The UVC reactor 10 is configured to turn healthy DNA 30 of any microorganisms in the water, such as bacteria, viruses, and other pathogens into damaged DNA 32.

Referring to FIG. 2, a second prior art UVC reactor 40 for treating water with UVC radiation 42 is illustrated. The UVC reactor 40 includes a reaction chamber 44, an inlet opening 46, an outlet opening 48, a first light source in the form of a plurality of LED packages 50 proximate to the inlet opening 46, and a second light source in the form of a plurality of LED packages 52 proximate to the outlet opening 48, configured to emit the UVC radiation 42. The location of the LED packages 50 provide longer exposure times for the water to the UVC radiation. This type of UVC reactor 40 is further described in U.S. Pat. No. 9,566,358.

Referring to FIGS. 3A and 3B, a third prior art UVC reactor 60 for treating water with UVC radiation 62 is illustrated. The UVC reactor 60 includes a first reaction chamber 64, having an inlet opening 66, and a second reaction chamber 68 having an outlet opening 70. The UVC reactor 60 also includes a light source in the form of an array of UVC LED emitters 72. A flow path 74 of the water through the reaction chambers 64, 68 is shown in FIGS. 3A and 3B. The flow path 74 through the reaction chamber 64, 68 provides longer exposure times for the water to the UVC radiation. This type of UVC reactor 60 is further described in U.S. Pat. No. 11,312,642.

Referring to FIG. 4, a fourth prior art UVC reactor 80 for treating water with UVC radiation 82 is illustrated. The UVC reactor 80 includes a housing 84 having a reaction chamber 94 with a plurality of flow-in openings 86 for the untreated water and a flow-out opening 88 for the treated water. The UVC reactor 80 also includes a UVC LED 90 configured to emit the UVC radiation 82 and a pump 92 for circulating the water. One problem with the UVC reactor 80 is that there are no reflective blockers for the UVC LED 90, such that UVC leakage can damage the housing 84 as well as biological entities in proximity to the UVC reactor 80.

The present disclosure is directed to a UV LED device configured to treat fluids in various applications. The present disclosure is also directed to a UVC reactor constructed with the UV LED device and to a method for treating fluids using the UV LED device.

### SUMMARY

A UV LED device for treating a fluid includes a housing having a reaction chamber, at least one inlet opening through the sidewalls of the housing in flow communication with the reaction chamber, and an outlet opening in flow communication with the reaction chamber. The housing of the UV LED device is configured for submersion in the fluid and is configured to direct fluid flow of untreated fluid from the inlet opening through the reaction chamber to the outlet opening. In addition, the housing is configured to direct the fluid flow of untreated fluid through different angles into the reaction chamber.

The UV LED device also includes a LED module mounted to the housing configured to emit UV radiation including radiation in the UVB wavelength range (280 nm to 310 nm) or the UVC wavelength range (200 nm to 280 nm). In addition, the LED module is configured to focus the UV radiation on the fluid flowing through the reaction chamber. The LED module can include a base, such as a PCB board or other circuitry substrate, at least one LED emitter mounted to the base configured to emit the UV radiation, and a sealed window in the reaction chamber for transmitting the UV radiation into the reaction chamber. The housing can also include a reflective coating on the walls of the reaction chamber configured to reflect the UV radiation onto the fluid, and a light blocker configured to prevent the UV radiation from exiting the reaction chamber. The UV LED device also includes a heatsink attached to the housing in thermal communication with the LED module and with the fluid in the reaction chamber. The heatsink, along with the fluid in the reaction chamber also being in thermal communication with the LED module, provides a self-cooling feature, with efficient heat transfer without the need for a separate cooling system.

A UV LED reactor includes a vessel configured to contain the fluid in flow communication with a source of the fluid. The UV LED reactor also includes the UV LED device submerged in the fluid contained within the vessel, and a flow generator for maintaining the fluid flow through the UV LED device. For example, the flow generator can be configured to maintain an untreated fluid flow through the inlet opening into the reaction chamber of the UV LED device for exposure to UV radiation, and a treated fluid flow through the outlet opening of the UV LED device for storage or use. The UV LED reactor also includes a power source for the LED module, which can comprise a hard-wired power source, or a wireless power source.

In an illustrative embodiment, the UV LED reactor is configured to treat water by sterilizing the water by exposure to UVB or UVC radiation, with the treated water discharged for storage or use. In an alternate embodiment, a UV LED fish tank reactor includes the UV LED device contained in a glass vessel and a recirculation loop for untreated and treated water. In another alternate embodiment, a UV LED air reactor includes the UV LED device and a fan configured to disinfect air by exposure to UVC radiation.

A method for treating a fluid includes the step of providing a UV LED reactor having a UV LED device submerged in the fluid that includes a housing having a reaction chamber, at least one inlet opening through the sidewalls of the housing in flow communication with the reaction chamber, and an outlet opening in flow communication with the reaction chamber, a LED module mounted to the housing configured to emit UV radiation including radiation in the UVB or UVC wavelength range, a light blocker on the housing within the reaction chamber configured to reduce radiation leakage through the inlet opening and the outlet opening, and a flow generator configured to generate a fluid flow through the UV LED device. The method also includes the step of directing untreated fluid from the inlet opening into the reaction chamber and exposing the untreated fluid to UV radiation in the reaction chamber. The method also includes the step of directing treated fluid from the reaction chamber into the outlet opening. The method can also include the step of providing the UV LED device further with a heatsink attached to the housing in thermal communication with the LED module, and the step of dissipating heat from the LED module using the heatsink.

### BRIEF DESCRIPTION OF THE DRAWINGS

All of the figures are schematic in nature and may not be drawn to scale.

FIG. 1 is a schematic cross-sectional view of a first prior art UVC reactor having a light source in the form of a UV tube lamp;

FIG. 2 is a schematic cross-sectional view of a second prior art UVC reactor having a first light source in the form of multiple LED emitters proximate to an inlet opening of a reaction chamber and a second light source in the form of multiple LED emitters proximate to an outlet opening of the reaction chamber;

FIG. 3A is a schematic cross-sectional view of a third prior art UVC reactor having multiple reaction chambers;

FIG. 3B is a schematic perspective view of the third prior art UVC reactor showing a flow path of water through the reaction chambers;

FIG. 4 is a schematic cross-sectional view of a fourth prior art UVC reactor having multiple inlet openings and a UVC LED having no reflective blocker;

FIG. 5A is a schematic perspective view a UV LED device having a LED module configured to emit UVB and UVC radiation and to treat fluids in various applications;

FIG. 5B is a cut away schematic perspective view of the UV LED device;

FIG. 6 is a schematic cross-sectional view of a UV reactor that includes the UV LED device configured to treat a fluid;

FIG. 7A is a schematic cross-sectional view of the UV LED device having the LED module mounted inside of the housing;

FIG. 7B is a schematic cross-sectional view of an alternate embodiment of the UV LED device having the LED module mounted outside of the housing;

FIG. 8A is a schematic cross-sectional view of the UV LED device having a plurality of circular openings in the housing;

FIG. 8B is a schematic side elevation view of the UV LED device shown in FIG. 8A;

FIG. 9A is a schematic side elevation view of an alternate embodiment UV LED device having a plurality of rectangular openings in the housing;

FIG. 9B is a schematic side elevation view of an alternate embodiment UV LED device having a single circular opening;

FIG. 9C is a schematic side elevation view of an alternate embodiment UV LED device having a single wide rectangular opening;

FIG. 9D is a schematic side elevation view of an alternate embodiment UV LED device having a single wide rectangular opening and a filter;

FIG. 10 is a schematic cross-sectional view illustrating components of a pumping system and circulation loop of an alternate embodiment UV LED fish tank reactor;

FIG. 11 is a schematic cross-sectional view of the alternate embodiment UV LED fish tank reactor with a wireless power supply;

FIG. 12A is a schematic cross-sectional view illustrating the circulation loop of the UV fish tank reactor with the inlet openings in the housing of the UV LED device functioning to receive untreated fluid;

FIG. 12B is a schematic cross-sectional view illustrating the circulation loop of the UV LED fish tank reactor with the inlet openings in the housing of the UV LED device functioning to discharge treated fluid;

FIG. 13A is a schematic side elevation view illustrating an alternate embodiment UV LED air reactor;

FIG. 13B is a schematic side elevation view illustrating an alternate flow path through the alternate embodiment UV LED air reactor;

FIG. 14A is a schematic perspective view illustrating the configuration of the cylindrical housing of the UV LED device;

FIG. 14B is a schematic perspective view illustrating the configuration of an alternate embodiment rectangular housing;

FIG. 14C is a schematic perspective view illustrating the configuration of an alternate embodiment horizontal cylindrical housing;

FIG. 15 is a schematic cross-sectional view illustrating the configuration of a light blocker of the UV LED device;

FIG. 16A is a schematic cross-sectional view illustrating a flow generator of the UV LED reactor in the form of an internal pump;

FIG. 16B is a schematic cross-sectional view illustrating a flow generator of the UV LED reactor in the form of an external pump;

FIG. 17A is a schematic cross-sectional view illustrating a flow generator of the UV LED reactor in the form of an air pump;

FIG. 17B is a schematic cross-sectional view illustrating a flow generator of the UV LED reactor in the form of a convection heater;

FIG. 18 is a schematic cross-sectional view illustrating a flow generator of the UV LED reactor in the form of a high potential vessel;

FIG. 19A is a schematic cross-sectional view illustrating a vortex flow generator of the UV LED reactor;

FIG. 19B is a schematic cross-sectional view of the vortex flow generator of the UV LED reactor taken along section line 19B of FIG. 19A; and

FIG. 20 is a schematic cross-sectional view illustrating an alternate embodiment vortex flow generator of the UV LED reactor.

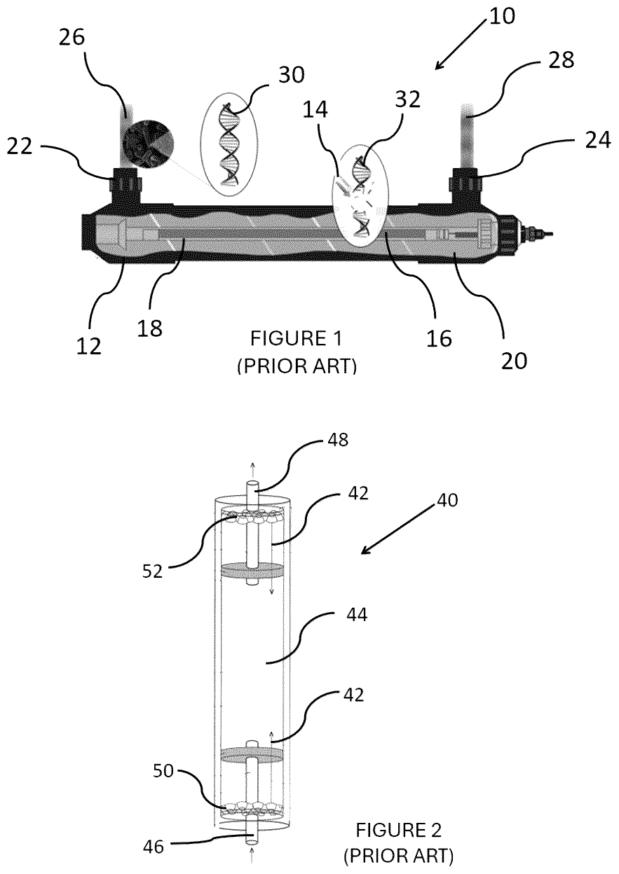

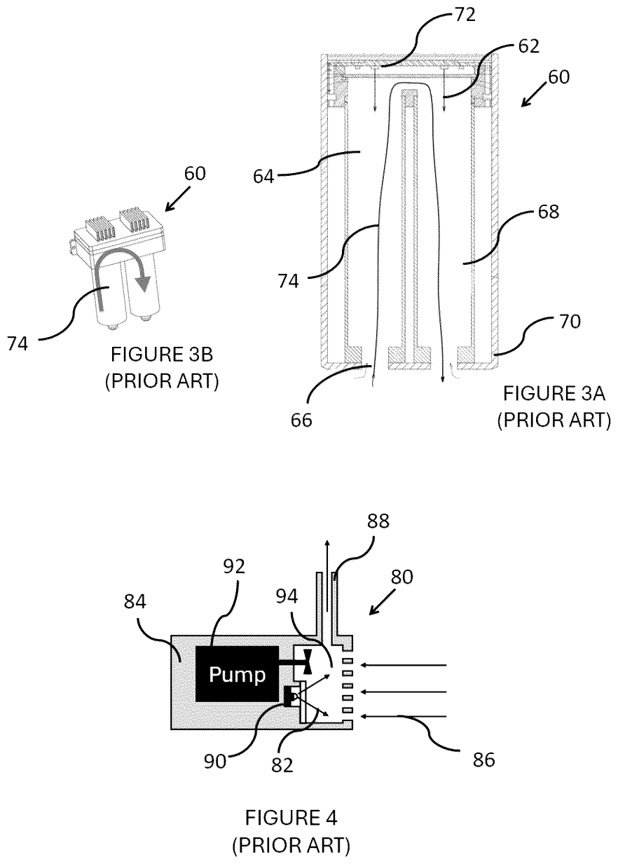

### DETAILED DESCRIPTION

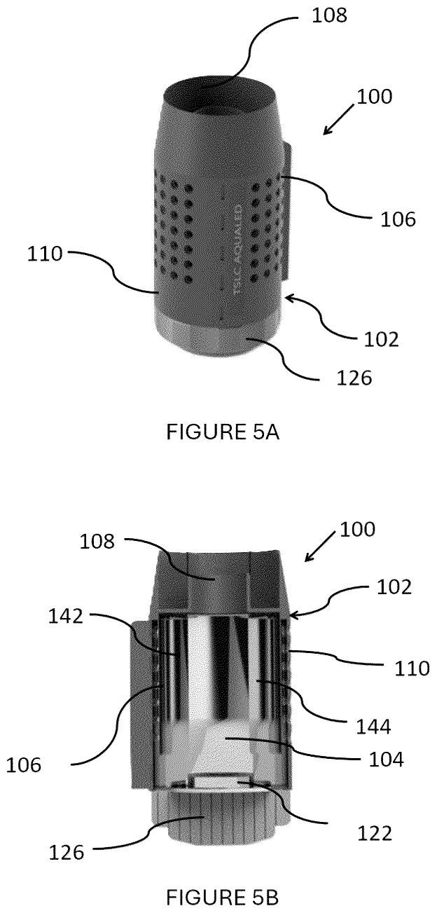

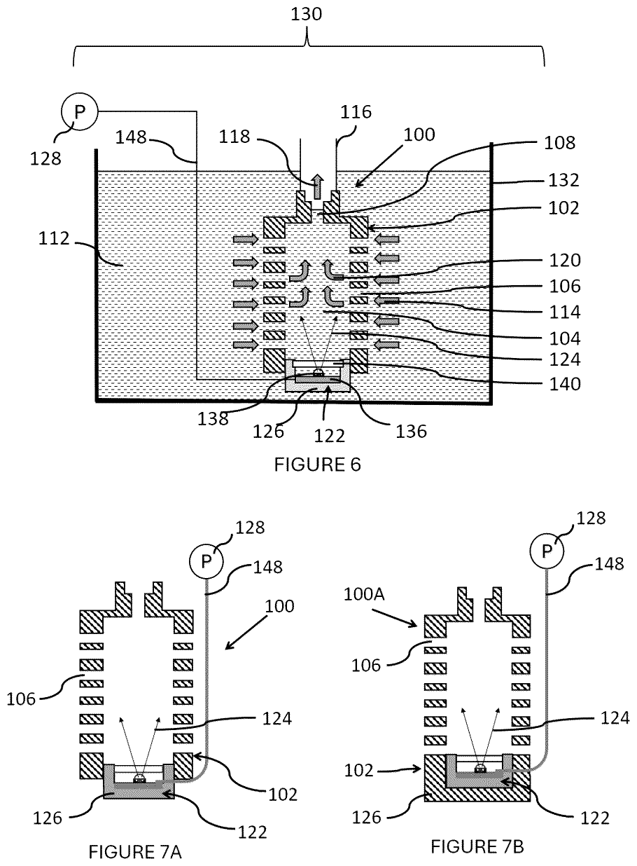

Referring to FIGS. 5A and 5B, a UV LED device 100 for treating a fluid 112 (FIG. 6) is illustrated. The UV LED device 100 includes a housing 102 having sidewalls 110, a reaction chamber 104 within the housing 102, a plurality of inlet openings 106 through the sidewalls 110 of the housing 102 in flow communication with the reaction chamber 104, and an outlet opening 108 in flow communication with the reaction chamber 104. The housing 102 can have sidewalls 110 constructed of a UV resistant material, such as a metal or a polymer. In addition, the housing 102 can have a desired geometrical configuration, such as cylindrical, rectangular, or square, with a hollow interior portion that forms the reaction chamber 104. Further, the housing 102 can comprise a unitary one-piece structure, or an assembly of different elements assembled into a unitary structure. Still further, the housing 102 can be manufactured using techniques that are known in the art such as molding and machining. A size of the housing 102, and a thickness of the sidewalls 110 of the housing 102, can be selected as required depending on the application.

As shown in FIG. 6, the UV LED device 100 also includes a LED module 122 configured to emit UV radiation 124, including radiation in the UVB wavelength range (280 nm to 310 nm) or the UVC wavelength range (200 nm to 280 nm). The LED module 122 includes a base 136, such as a PCB board or other circuitry substrate, at least one LED emitter 138 mounted to the base 136 configured to emit the UV radiation 124, and a scaled window 140 in physical contact and thermal communication with the fluid 112 in the reaction chamber 104, configured to seal the LED emitter 138 from the fluid 112, and to transmit the UV radiation 124 into the reaction chamber 104 to treat the fluid 112. The sealed window 140 can comprise a UV transparent material, such as quartz, fused silica or a UV transparent polymer, having a desired peripheral shape, such as a peripheral shape that matches the peripheral shape of the housing 102 (e.g., circular, square, rectangular).

Preferably, the LED module 122 is removable from the housing 102 as a replaceable component. This allows different wave lengths of UV to be used for performing different treatment processes. In addition, the LED module 122 can be include different types of LED emitters 138, such as one or more LED emitters 138 configured to emit UV radiation having a wavelength range of from 280 nm to 310 nm (UVB), in combination with one or more LED emitters 138 configured to emit UV radiation having a wavelength range of from 200 nm to 280 nm (UVC), or any other selected UV wavelength range. In addition, the LED module 122 is configured to focus the UV radiation 124 (FIG. 6) on the fluid 112 flowing through the reaction chamber 104. The housing 102 can also include a reflective material 142 (FIG. 5B) on the sidewalls 110 of the reaction chamber 104 configured to reflect all of the UV radiation 124 (FIG. 6) emitted by the LED emitter 138 onto the fluid 112. The LED module 122 also includes a light blocker 144 within the reaction chamber 104 configured to block the UV radiation 124 emitted by the LED emitter 138 including reflected UV radiation 124, from exiting the reaction chamber 104. The light blocker 144 preferably comprises a reflective material having a greater than 35% UV radiation reflectivity. Suitable materials for the light blocker 144 include aluminum, fluorinated polymers, plastics, ceramics and glass.

The UV LED device 100 also includes a heatsink 126 attached to the housing 102 in thermal communication with the LED module 122 configured to dissipate heat generated by the LED module 122. In the UV LED device 100, the heatsink 126 is in contact with the housing 102, and also includes one or more surfaces in contact with the fluid 112 flowing within the reaction chamber 104. In addition, the UV LED device 100 is in thermal communication with the fluid 112, as the window 140 of the LED module 122 is in direct physical contact with the fluid 112 in the reaction chamber 104. This arrangement effectively removes the heat from the LED emitter 138 without the need for an additional cooling fan. The heatsink 126 can comprise a material having a high thermal conductivity, such as metal, a heat-dissipating plastic or a ceramic. A thermal conductivity coefficient of the heatsink 126 is preferably more than 20 W/mK. In addition, the heatsink 126 preferably comprises a single piece of material with a continuous surface in contact with the fluid 112 flowing within reaction chamber 104 to provide efficient cooling performance. The materials for the heatsink 126 can be selected to ensure efficient heat transfer and to maintain the LED emitter 138 at an optimal operating temperature, thereby enhancing performance and longevity without the requirement of a separate cooling system as in the prior art. In addition, the heatsink 126 can have a desired size and peripheral shape, such as circular, square or rectangular to match the peripheral shape of the housing 102. The sealed window 140 of the LED module 122 can be attached to a groove in the heatsink 126 using a UV resistant sealant such as an adhesive polymer, and O-rings if necessary.

Referring to FIG. 6, a UV LED reactor 130 constructed using the UV LED device 100 is illustrated. The UV LED reactor 130 includes a vessel 132 configured to contain a desired quantity of the fluid 112 in untreated form. The vessel 132 can comprise a rigid material such as metal, plastic or glass. The vessel 132 can be in flow communication with a source (not shown) of the fluid 112. In addition, the vessel 132 can comprise a sealed or unsealed vessel having a desired size and geometrical configuration. The UV LED device 100 is configured for submersion in the fluid 112 contained within the vessel 132.

The UV LED reactor 130 also includes a power source 128 in electrical communication with the LED module 122 via a hard wire 148. In addition, the UV LED reactor 130 is configured to maintain an untreated fluid flow 114 through the inlet openings 106 into the reaction chamber 104 for treatment by UV as indicated by a UV exposed fluid flow 120. The outlet opening 108 of the housing 102 is configured for attachment to an outlet conduit 116 for receiving a treated fluid flow 118, which can be stored in a separate vessel (not shown) or used as required. The untreated fluid flow 114, the UV exposed fluid flow 120 and the treated fluid flow 118 can be generated externally or internally by a suitable flow generator, such as a pump, a motor, a pressure differential arrangement, a potential differential arrangement, an air generator, a bubble generator, a thermal convection device, thermal flow generated by the LED module 122, or any other mechanism operably associated with the UV LED device 100.

As shown in FIG. 7A, the LED module 122 can be mounted on the outside of the housing 102. Alternately, as shown in FIG. 7B, the LED module 122 can be mounted on the inside of the housing 102.

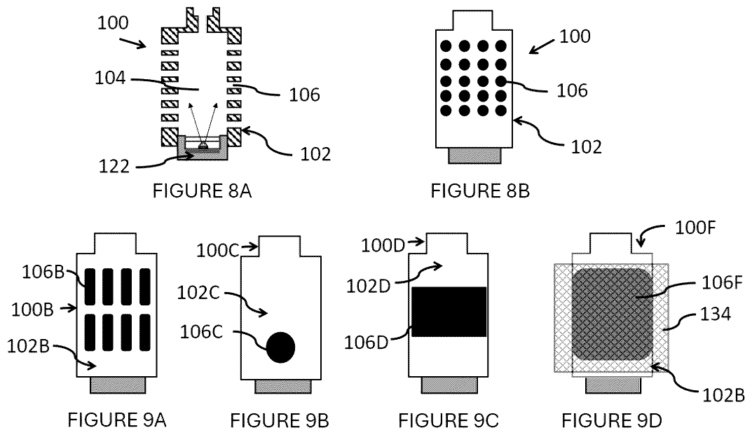

As shown in FIGS. 8A and 8B, the inlet openings 106 can comprise circular shaped openings that are arranged in a geometrical pattern along the circular outside surface of the housing 102. With the housing 102 having a generally cylindrical shape, the inlet openings 106 direct an untreated fluid flow 114 in all directions into the reaction chamber 104. This provides an efficient structure for treating the fluid 112 without the necessity of complex flow structures as in the prior art.

FIGS. 9A-9D illustrate alternate embodiment LED devices 100B-100F. In FIG. 9A, a LED device 100B includes a housing 102B having a plurality of rectangular shaped inlet openings 106B. In FIG. 9B, a LED device 100C includes a housing 102C having a single large circular inlet opening 106C. In FIG. 9C, a LED device 100D includes a housing 102D having a single large rectangular or square inlet opening 106D. In FIG. 9D, a LED device 100F includes a housing 102F having a single large circular inlet opening 106F covered by a filter element 134.

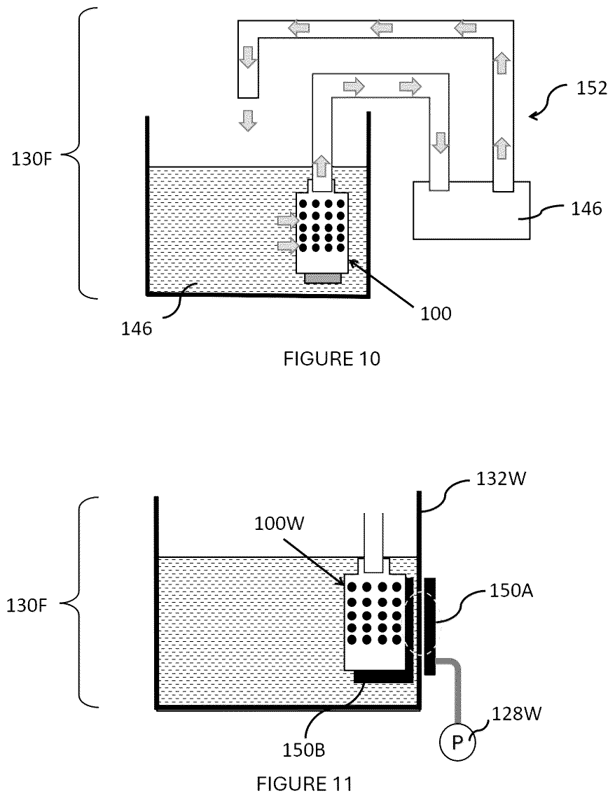

FIG. 10 illustrates an alternate embodiment UV LED fish tank reactor 130F. FIG. 10 also illustrates the flow paths to and from the UV LED fish tank reactor 130F using a flow generator 146 in the form of a pump, and a recirculation loop 152 for the fluid 112.

FIG. 11 illustrates an alternate embodiment UV LED device 100W having a wireless power supply 128W in electrical communication with a first coil 150A external to the vessel 132W. In addition, the first coil 150A is in electromagnetic communication with a second coil 150B on the UV LED device 100W. In this embodiment, the vessel 132W preferably comprises a material, such as glass, which allows the electromagnetic fields to couple between the coils 150A, 150B using techniques that are known in the art, such as resonance coupling. This embodiment is particularly suited for use with the UV LED fish tank reactor 130F.

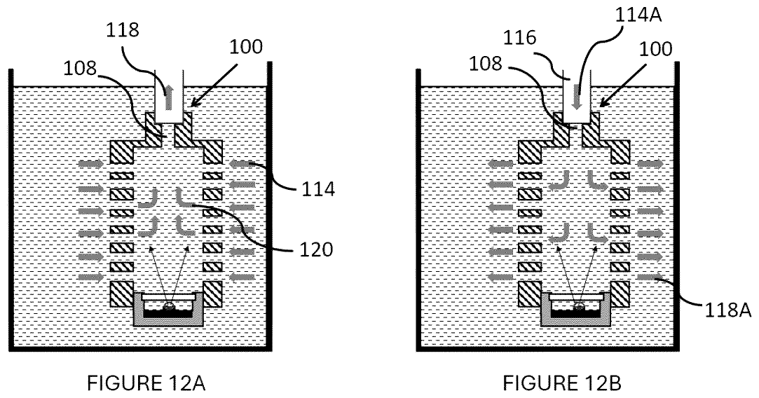

FIG. 12A illustrates the preferred flow path for the UV LED reactor 130 in which untreated fluid flow 114 is directed through the inlet openings 106 and the treated fluid flow 118 is directed through the outlet opening 108. However, as shown in FIG. 12B, an alternate untreated fluid flow 114A is directed through the outlet opening 108 and a treated fluid flow is directed through the inlet openings 106.

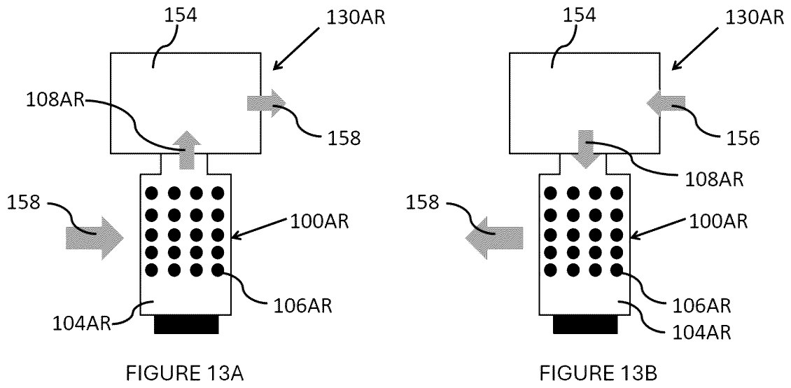

Referring to FIGS. 12A and 12B, a UV LED air reactor 130AR includes a UV LED device 100AR and a fan 154 configured to move air through a reaction chamber 104AR for disinfection, substantially as previously described for UV LED reactor 130. In FIG. 13A, untreated air 156 is moved by the fan 154 through the inlet openings 106AR for treatment in the reaction chamber 104AR and treated air 158 is discharged through the outlet opening 108AR. In FIG. 13B, untreated air 156 is moved by the fan 154 through the outlet opening 108AR for treatment in the reaction chamber 104AR and treated air 158 is discharged through the inlet openings 106AR.

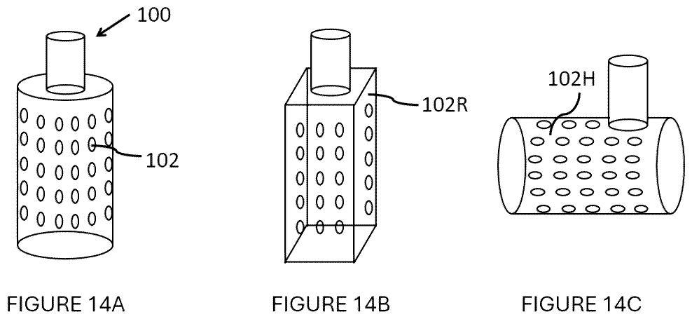

Referring to FIGS. 14A-14C, different configurations for the housing of the UV LED device 100 are illustrated. FIG. 14A illustrates the previously described cylindrical housing 102 of the UV LED device 100. FIG. 14B illustrates an alternate embodiment rectangular or square housing 102R. FIG. 14C illustrates an alternate embodiment horizontal cylindrical housing 102H.

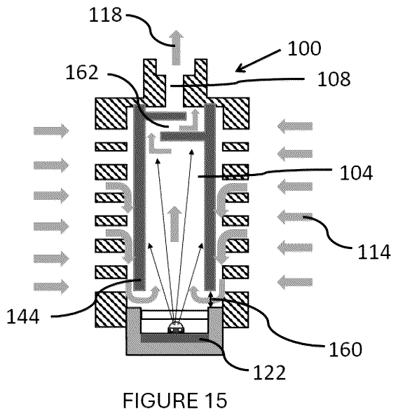

Referring to FIG. 15, an exemplary configuration for the light blocker 144 is illustrated. In this example, the light blocker 144 has the configuration of a baffle. The light blocker 144 is configured to minimize UV radiation leakage from the reaction chamber 104 and to provide protection for human eyes, fish or other species. The light blocker 144 forms a gap 160 between the LED module 122 and the light blocker 144 configured to allow the untreated fluid flow 114 to enter the reaction chamber 104 substantially as shown in FIG. 15. In addition, a baffle channel 162 prevents the UV radiation 124 from exiting the reaction chamber 104, while allowing treated fluid flow 118 to be discharged from the discharge opening 108. The light blocker 144 can comprise a UV-resistant material and a UV-reflective material as well, including suitable polymers, plastics, ceramics, glasses or metals. In addition, the light blocker 144 can include a coating or plating on the surface thereof to facilitate UV resistance and UV reflection. As will be further explained, the light blocker 144 can also be configured as a vortex flow generator.

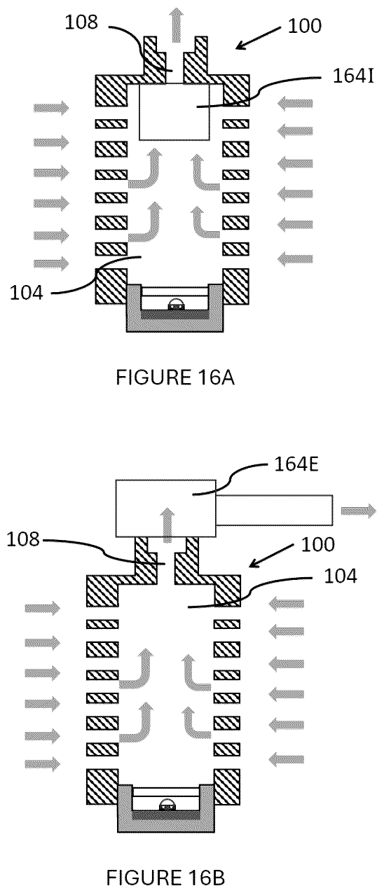

Referring to FIGS. 16A-16B, exemplary configurations for a flow generator of the UV LED reactor 130 are illustrated. In FIG. 16A, the flow generator comprises an internal pump 164I mounted within the reaction chamber 104 in flow communication with the outlet opening 108, substantially as shown. In FIG. 16B, the flow generator comprises an external pump 164E mounted externally to the reaction chamber 104 in flow communication with the outlet opening 108 substantially as shown.

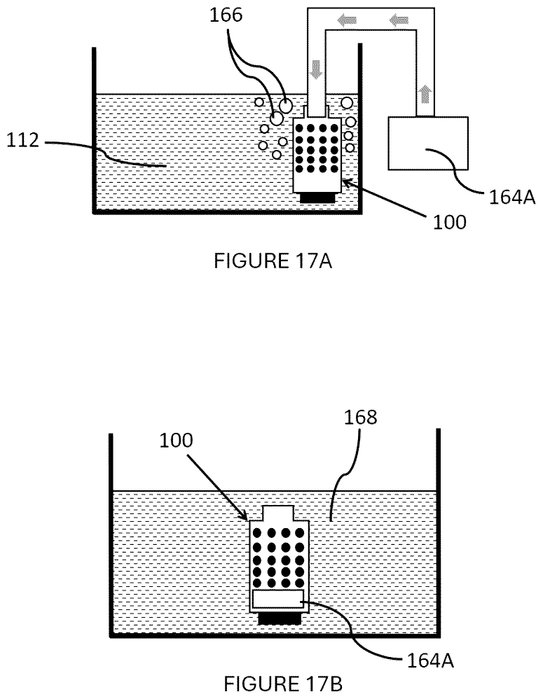

Referring to FIGS. 17A-17B, additional exemplary configurations for a flow generator of the UV LED reactor 130 are illustrated. In FIG. 17A, the flow generator comprises an air pump 164A configured to inject air bubbles 166 for maintaining a desired fluid flow through the LED device 100. In FIG. 17B, the flow generator comprises a convection heater 164H configured to generate a thermal flow path 168 through the LED device 100.

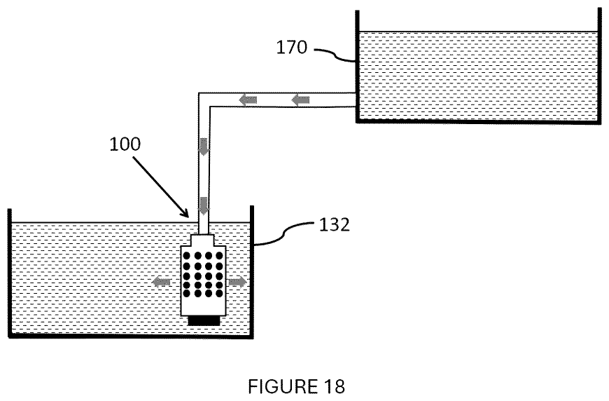

Referring to FIG. 18, an additional exemplary configuration for a flow generator of the UV LED reactor 130 is illustrated. In FIG. 18 the flow generator comprises a high potential vessel 170 operably associated with the low potential vessel 132 in which the LED device 100 is submerged. In this embodiment, fluid flow occurs from the high potential vessel 170 to the low potential vessel 132 substantially as shown in FIG. 18.

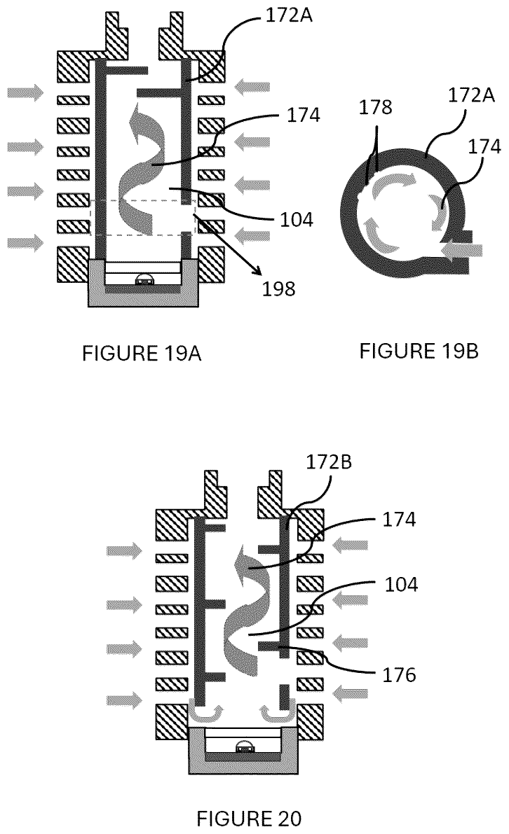

Referring to FIGS. 19A and 19B, an additional exemplary configuration for a vortex flow generator 172A of the UV LED reactor 130 is illustrated. The vortex flow generator 172A has a baffle configuration and is configured to generate a vortex flow 174, by its circular cross-section, substantially as shown in FIGS. 19A and 19B. The vortex flow 174 increases the dwell time of the fluid 112 in the reaction chamber 104 and facilitates exposure of molecules and microorganisms in the fluid 112 to the UV radiation 124. In addition, the vortex flow generator 172A can be configured as a UV light blocker as previously described for light blocker 144 (FIG. 15). Further, the vortex flow generator 172A can include surface features 178, such as ridges and grooves, configured to enhance spiral flow pattern of the vortex flow 174.

Referring to FIG. 20, an additional exemplary configuration for a vortex flow generator 172B of the UV LED reactor 130 is illustrated. The vortex flow generator 172B includes a plurality of vortex flow ribs 176 in a spiral configuration, configured to generate a vortex flow 174 substantially as shown in FIG. 20. In addition, the vortex flow generator 172B can be configured as a UV light blocker as previously described for light blocker 144 (FIG. 15).

While a number of exemplary aspects and embodiments have been discussed above, those of skill in the art will recognize certain modifications, permutations, additions and subcombinations thereof. It is therefore intended that the following appended claims and claims hereafter introduced are interpreted to include all such modifications, permutations, additions and sub-combinations as are within their true spirit and scope.

What is claimed is:

1. A UV LED device for treating a fluid comprising;
a housing configured for submersion in the fluid, the housing comprising a reaction chamber, at least one inlet opening configured to allow flow of untreated fluid into the reaction chamber, and at least one outlet opening configured to allow flow of a treated fluid out of the reaction chamber;
a LED module mounted to the housing comprising at least one UV LED emitter configured to emit UV radiation into the reaction chamber and a window in the reaction chamber configured to seal the UV LED emitter from the fluid; and
a light blocker on the housing within the reaction chamber configured to reduce radiation leakage through the inlet opening and the outlet opening.

2. The UV LED device of claim 1 wherein the reaction chamber includes sidewalls comprising a UV resistant or UV reflective material.

3. The UV LED device of claim 1 wherein the at least one inlet opening comprises a plurality of openings arranged along a periphery of the housing configured to direct the flow of untreated fluid into the reaction chamber at different angles.

4. The UV LED device of claim 1 further comprising a heatsink attached to the housing in thermal communication with the LED module.

5. The UV LED device of claim 1 wherein the light blocker comprises a reflective material having a greater than 35% UV radiation reflectivity.

6. The UV LED device of claim 1 wherein the light blocker comprises a material selected from the group consisting of aluminum, fluorinated polymers, plastics, ceramics and glass.

7. The UV LED device of claim 1 wherein the light blocker is configured to generate a vortex flow path of the fluid through the reaction chamber.

8. The UV LED device of claim 1 further comprising a filter on the at least one inlet opening configured to filter the untreated fluid.

9. The UV LED device of claim 1 further comprising a power supply in electrical communication with the LED module.

10. The UV LED device of claim 1 further comprising a coil on the housing configured for electromagnetic communication to a wireless power supply for the LED module.

11. The UV LED device of claim 1 wherein a UV radiation wavelength range of the at least one LED emitter of the LED module is between 280 nm to 310 nm (UVB) or between 200 nm to 280 nm (UVC).

12. The UV LED device of claim 1 wherein the fluid comprises water.

13. The UV LED device of claim 1 wherein the fluid comprises air.

14. The UV LED device of claim 1 wherein the housing has a shape selected from the group consisting of cylindrical, rectangular and square.

15. The UV LED device of claim 1 wherein the window of the LED module is in direct physical contact with the fluid in the reaction chamber and comprises a material selected from the group consisting of quartz, fused silica, and UV transmissive polymers.

16. A UV LED reactor for treating a fluid comprising:
a vessel configured to contain the fluid;
a UV LED device submerged in the fluid contained within the vessel, the UV LED device comprising a housing comprising a reaction chamber, at least one inlet opening configured to allow flow of untreated fluid into the reaction chamber, and at least one outlet opening configured to allow flow of a treated liquid out of the reaction chamber, and an LED module mounted to the housing comprising at least one UV LED emitter configured to emit UV radiation into the reaction chamber and a window in direct physical contact with the fluid in the reaction chamber configured to seal the UV LED emitter from the fluid;
a light blocker on the housing within the reaction chamber configured to reduce radiation leakage through the inlet opening and the outlet opening; and
a flow generator configured to generate a fluid flow through the UV LED device.

17. The UV LED reactor of claim 16 wherein the flow generator comprises an element selected from the group consisting of a pump, a motor, a pressure differential arrangement, a potential differential arrangement, an air generator, a bubble generator, a thermal convection device, a thermal flow generated by the LED module.

18. The UV LED reactor of claim 16 further comprises a wireless power source in electromagnetic communication with the LED module.

19. The UV LED reactor of claim 16 wherein the fluid comprises water and a UV radiation wavelength range of the at least one LED emitter of the LED module is between 280 nm to 310 nm (UVB) or between 200 nm to 280 nm (UVC).

20. The UV LED reactor of claim 16 wherein the fluid comprises air and the flow generator comprises a fan attached to the housing.

21. The UV LED reactor of claim 16 wherein the reaction chamber of the UV LED device includes sidewalls comprising a UV resistant or UV reflective material.

22. The UV LED reactor of claim 16 wherein the at least one inlet opening comprises a plurality of openings arranged along a periphery of the housing of the UV LED device configured to direct the flow of untreated fluid into the reaction chamber at different angles.

23. The UV LED reactor of claim 16 further comprising a heatsink attached to the housing in thermal communication with the LED module.

24. The UV LED reactor of claim 16 wherein the vessel comprises a fish tank and the flow generator comprises a pump and a recirculation loop for the fluid.

25. A method for treating a fluid comprising:
providing a UV LED reactor having a UV LED device submerged in the fluid that includes a housing having a reaction chamber, at least one inlet opening through the sidewalls of the housing in flow communication with the reaction chamber, and an outlet opening in flow communication with the reaction chamber, a LED module mounted to the housing configured to emit UV radiation including radiation in the UVB or UVC wavelength range, a light blocker on the housing within the reaction chamber configured to reduce radiation leakage through the inlet opening and the outlet opening, and a flow generator configured to generate a fluid flow through the UV LED device;
directing untreated fluid from the inlet opening into the reaction chamber and exposing the untreated fluid to UV radiation in the reaction chamber;
directing treated fluid from the reaction chamber into the outlet opening; and
reducing radiation leakage from the LED module using the light blocker.

26. The method of claim 25 wherein the at least one inlet opening comprises a plurality of openings arranged along a periphery of the housing configured to direct the flow of untreated fluid into the reaction chamber at different angles.

27. The method of claim 25 wherein the UV LED device further comprises a heatsink attached to the housing in thermal communication with the LED module and further comprising dissipating heat from the LED module using the heatsink.

28. The method of claim 25 wherein UV LED reactor includes a vessel configured to contain the fluid in the form of water.

29. The method of claim 25 wherein the fluid comprises air and further comprising providing the UV LED reactor with a fan configured to move the air from the at least one inlet opening to the outlet opening.

30. The method of claim 25 wherein the UV LED reactor includes a vessel configured as a fish tank.

31. The method of claim 25 wherein the fluid comprises water and a UV radiation wavelength range of the at least one LED emitter of the LED module is between 280 nm to 310 nm (UVB) or between 200 nm to 280 nm (UVC).

32. The method of claim 25 wherein the housing has a shape selected from the group consisting of cylindrical, rectangular and square.

33. The method of claim 25 wherein the LED module includes a window in direct physical contact with the fluid in the reaction chamber and the window of the LED module comprises a material selected from the group consisting of quartz, fused silica, and UV transmissive polymers.
